(19) United States
(12) Patent Application Publication
(10) Pub. No.: US 2004/0170714 A1
(43) Pub. Date: Sep. 2, 2004

(54) Bauhinia extracts

(76) Inventors: Herwig Buchholz, Frankfurt (DE); Corinna Wirth, Darmstadt (DE); Valerie Bicard-Benhamou, Darmstadt (DE); Deoclecio Carmo, Rio de Janeiro (BR); Didier Mesangeau, Combs la Ville (FR)

Correspondence Address: Millen White, Zelano & Branigan, Arlington Courthouse Plaza I, 2200 Clarendon Boulevard Suite 1400, Arlington, VA, 22201, US

(21) Appl. No.: 10/484,903

(22) PCT Filed: Jul. 1, 2002
(86) PCT No.: PCT/EP02/07218

(30) Foreign Application Priority Data
Jul. 27, 2001 (EP) 01117711.0

(51) Int. Cl. A61K 35/78

(52) U.S. Cl. 424/774

(57) ABSTRACT

The present invention relates to an plant extract from Bauhinia species with hypoglycaemic activity, which is characterized in being obtained from a Bauhinia species and to a method for producing the extract and the use of the extract in the treatment of diabetes type 2.

The present invention relates to an plant extract with hypoglycaemic activity, a method for producing the extract and the use of the extract in the treatment of diabetes.

Diabetes mellitus is the only non-infectious disease designated as an epidemic by the World Health Organization (“Prevention of Diabetes Mellitus”, World Health Organization Technical Report Series, No. 844 (1994)). The prevalence of all types of diabetes is estimated to be 2.3% of the world's population, with the number of diabetics increasing by 4-5% per annum. It is projected that as many as 40-45% of persons aged 65 or older have either Type 2 diabetes or its precursor state, impaired glucose tolerance (IGT). In the US˜10% of the diabetic population suffer from Type 1 diabetes, an autoimmune disease characterized by the loss of pancreatic β-cell function and an absolute deficiency of insulin. The remainder of the diabetic population suffers from Type 2 diabetes or IGT, which although related to the body's inability to properly respond to insulin, have a more complex etiology (American Diabetes Association, Diabetes Care, 22 (Suppl. 1), S27 (1999). Diabetes can be treated by a combination of lifestyle change and medication. However, the metabolic disorder underlying diabetes also affects protein and lipid metabolism, leading to serious complications, including peripheral nerve damage, kidney damage, impaired blood circulation, and damage to the retina of the eye. Diabetes is the leading cause of blindness and amputation in western populations, and the direct medical costs alone were estimated to be ˜$44bn in the US alone in 1998 (Am. Diabetis Association, Diabetis Care 22(Suppl.1), S27 (1999)).

The United Kingdom Prospective Diabetes Study (UKPDS), a long-term study of Type 2 diabetics, has shown that rigorous management of blood glucose levels (measured as hemoglobin, HbA_{1c}), and blood pressure substantially reduce the incidence of complications (R. C. Turner, C. A. Cull, V. Frighi, R. R. Holman J. Am Med. Assoc., 281 2005 (1999)). The current therapeutic strategies for Type 2 diabetes are limited, and involve insulin therapy and oral hypoglycemic agents (OHAs) such as sulfonylureas, metformin, and the thiazolidinediones. Combination therapy with one or more of these agents is now a viable option as target blood glucose levels become harder to maintain with monotherapy (R. C. Turner, C. A. Cull, V. Frighi, R. R. Holman J. Am Med. Assoc., 281 2005 (1999); UK Prospective Diabetes Group, Lancet, 352, 837 (1998)).

On the other hand plants have been sources of medication since ancient times and many ethnic groups have favorite remedies, real and imaginary, for diabetes. Synthetic drugs or isolated natural products have largely replaced medicinal plants and their extracts in therapeutic use. Examples of international studies of plants with hypoglycemic effect include: blueberry leaves (Allen, 1927), roots of Fatsia horrida, Pterocarpus marsupium, Eugenia jambolana and Aspergicus niger (Krall, 1971), Aloe barbadensis and Opunthia streptacantha (Frati-Mnuari et al., 1988).

In E. M. K. Russo, A. A. J. Reichelt, J. R. De-Sá, R. P. Furlanetto, R.C.S. Moisés, T. S. Kasamatus and A. R. Chacra; “Clinical trial of Myrcia uniflora and Bauhinia fortificata leaf extracts in normal and diabetic patients”, Brazilian J Med Bio Res (1990) 23: 11-20; Myrcia uniflora and Bauhinia fortificata were compared with placebo for their hypoglycemic effect in randomized cross-over double-blind studies in 2 groups of normal subjects (10 subjects each) and 2 groups of Type II diabetic patients (18 in the M. uniflora group and 16 in the B. fortificata group). The protocol with each plant lasted 56 days. After the ingestion of infusions of 3 g leaves/day in water of M. uniflora and B. fortificata leaves, no acute or chronic effects on plasma glucose levels or glycated hemoglobin were found in either group. However, plasma insulin levels in the diabetic group were lower after M. uniflora than after placebo. There were no differences in any clinical parameters after the use of placebo or of B. fortificata. It is concluded that infusions prepared from the leaves of M. uniflora or B. fortificata have no hypoglycemic effect on normal subjects or Type II diabetic patients, there.

There is still demand for oral hypoglycemic agents useful in therapy for Type 2 diabetes.

We surprisingly found, that Extracts from Bauhinia species show hypoglycemic activity and are useful as oral hypoglycemic agents.

A first embodiment of our invention is therefore an plant extract with hypoglycaemic activity, characterized in that it is obtained from a Bauhinia species.

Preferred extracts are characterized in that the area under the curve—Plasma glucose concentration vs. Time—, in the Oral Glucose Tolerance Test according to the N0-STZ Rat model decreases significantly.

The N0-STZ rat model is described in Portha B., Picon L., Rosselin G., Diabetologia, 1979, 17, 371-377. The Oral Glucose Tolerance Test (OGTT) is described in Wilkerson: Diagnosis, oral glucose tolerance test: in Diabetes mellitus, Diagnosis & Treatment, p.31-34, NY, American Diabetes Association, 1964. During 5 days an appropriate dose of the extract is gavaged into the N0-STZ rats two times a day. Three hours after the last administration the Oral Glucose Tolerance Test (OGTT) is performed in awake rats by administering 2 g glucose/kg body weight. Blood samples are collected just before glucose administration (0 min) and after 30, 60 90, 120 and 180 minutes.

Preferred extracts of our invention show a decrease in the area under the curve plasma glucose concentration versus time of at least 10%, preferable at least 15%, even more preferred at least 20% and most preferred at least 35% versus control.

It is fUrthermore preferred, that the extract, when applied to rats according to the N0-STZ Rat model, decreases the fasting Plasma Glucose Concentration versus initial Basal Glycemia significantly. The fasting plasma glucose concentration (table 4) is measured from blood samples obtained after a 2 hour fasting period on day 5 before the administration of glucose. In addition, preferred extracts of the invention in hand show a significant decrease of the fasting plasma glucose concentration versus initial basal glycemia. Preferred extracts show a decrease of the fasting plasma glucose concentration of at least 10%, more preferred of at least 28% versus initial basal glycemia.

The extract of our invention can be obtained from any Bauhina species, of which are known the species: Bauhinia candicans, Bauhinia championii, Bauhinia fortificata (=Bauhinia forficata), Bauhinia manca, Bauhinia purpurea, Bauhinia racemosa, Bauhinia reticulatea, Bauhinia tomentosa, Bauhinia variegate, Bauhinia cheilantha, Bauhinia guanensis, Bauhinia refusa, Bauhinia glauca, Bauhinia pauletia, Bauhinia ungulato, Bauhinia macrostachya and Bauhinia splendens. Preferably the extract is obtained from Bauhinia fortificata (=Bauhinia forficata).

In principle all aerial parts of the plants may be used for the extract. The best results were obtained for extracts from the leaves, especially young leaves of Bauhinia. Therefore extracts from young leaves are especially preferred in our invention.

Our investigations indicate, that the high activity of the Bauhina extracts is due to a combination of various ingredients of the extract.

Components which were identified from the extracts are:

from Bauhinia candicans: rutin, quercetin, quercitrin, isoquercetin (isoquercitrin), campesterol, stigmasterol, cholesterol, stigmast-3,5-dien-7-one, triacontanol, cholin, trigonellin, trigonellin acetate, kaempferol-3-rutinoside, kaempferol-3-rutinoside-7-rhamnoside, sitosterol-3-glycoside (aerial parts), sitosterol-3-O-β-D-xylopyranoside, sitosterol-3-O-α-D-ribunonosofuranoside , 3-O-methyl-D-insoitol (D-Pinit), sitosterol 3-O-D-xyluronofuranoside;

from Bauhinia championii: 5,6,7,5′-tetramethylenedioxy-3′,4′,-methylendioxyflavone, 5,6,7,5,3′,4,5′-hexamethoxyflavone, 5,7,5′-trimethoxy-3′,4 ′,-methylenedioxyflavone;

from Bauhinia fortificata: quercetin, quercetin-3,7-O-α-dirhamnoside, isoquercetin (isoquercitrin), kaempferol-3-rutinoside, rutin, quercitrin, campesterol, stigmasterol, cholesterol, stigmast-3,5-dien-7-one, triacontanol, cholin, trigonellin, kaempferol, kaempferol-7-O-α-rhamnoside, kaempferol-3-rutinoside-7-rhamnoside, kaempferol 3,7-dirhamnoside (kaempferitrin), sitosterol-3-glycoside (aerial parts), 3-O-methyl-D-inositol (D-pinit), beta-sitosterol, daucosterol, lupeol, saponins, tannins, astragalin;

from Bauhinia manca: p-cumaric acid, ferulic acid, phytosterols, cinnamic. acid, gallic acid, epicatechin -3-gallate, 5,7-dihydroxychromon, hydroxypropioguaiacon, obtustyren, isoliquitigenin4-methylether, liquiritigenin-4′-methylether, 2,4′-dihydroxy4-methoxydihydrochalcon, 4′-hydroxy-7,3′-dimethoxy-flavan, 3′,4′-dihydroxy-7-methoxyflavan, syringaresinol, 5,5′-dimethoxylariciresinol, chrysoeriol, luteolin-5,3′-dimethylether;

from Bauhinia purpurea: 6′-(stigmast-5-en-7-one-3-O-glucopyranosidyl)-hexadecanoate, 3-hydroxystigmast-5-en-7-one, Oleanolic acid, 6,8-dimethylchrisin, Chrysin, Isoquercetin (Isoquercitrin), Astragalin, 2,3-dihydroxyproply-oleate, 2,3-dihydroxypropyllinoleate, 2,3-dihydroxypropyl-16-hydroxyhexadecanoate, 6-butyl-3-hydroxyflavavone (6-(3″-oxobutyl)-taxifolin, 5,6-dihydroxy-7-methoxyflavone 6-O-D-xylopyrynose;

from Bauhinia racemosa: kaempferol, quercetin, kaempferol-3-O-rhamnoside, quercitrin, D-O-methylracemosol, pacharin;

from Bauhinia reticulata: quercetin, quercitrin;

from Bauhinia tomentosa: rutin, quercetin, isoquercetin (isoquercitrin);

from Bauhinia variegate: apigenin, apigenin-7-O-glucoside, kaempferol-3-galactoside, kaempferol-3-rhamno-glycoside, kaempferol-3-glucoside (astragalin), sitosterol, lupeol, naringenin-4′-rhamnoglucoside, naringin 5,7-dimethyl-ether4′-rhamnoglycoside, 5,7-dihydroxyflavonanone4′-O-L-rhamnopyranosyl-β-D-glucopyranoside, (naringenin aglycone), from hydrolysis results: Quercetin in leaves, flowers, seeds, myricetin in seeds, dihydroxyquercetin (taxifolin) perikarp, kaempferol flowers, methyl esters of kaempferol in flowers, methyl esters of quercetin in leaves, taxifolin, rutin, quercetin, quercitrin, isoquercetin (isoquercitrin);

from Bauhinia guanensis (in the stem bark): beta-sitosterol, stigmasterol, 3-O-glucopyranosylstigmasta-5-22-diene, 3-O-glucopyranosyl-sitostreol, 4′-hydroxy-7-methoxyflavan, lapachol.

Our results show a strong hypoglycemic activity of the extracts of our invention. The effect is comparable to effects obtained with pure actives such as mefformin.

We assume that the strong effects of the extracts are due to a combination of at least two different actives. At least one of the actives is supposed to be a flavone or flavonoid, such as apigenin, apigenin-7-O-glucoside, isoquercetin (isoquercitrin), kaempferol-3-rutinoside, kaempferol-3-galactoside, kaempferol3-rhamno-glycoside, kaempferol-3-glucoside (astragalin), naringenin4′-rhamnoglucoside, naringin, quercetin, quercetin-3,7-O-α-dirhamnoside, quercitrin, 5,7-dimethyl-ether-4′-rhamnoglycoside, rutin, 5,7-dihydroxyflavanone4′-O-L-rhamnopyranosyl-β-D-glucopyranoside (naringenin aglycone), preferably at least one active is selected from quercetin, isoquercetin or rutin.

Studies on isoquercetin show for this active alone a measurable hypoglycemic activity. Therefore the use of Isoquercetin for the production of a medicament with hypoglycaemic activity is an embodiment of the invention in hand.

Further ingredients of the Bauhinia extracts include polypeptides, polysaccharides, steroids and saponins. In a preferred embodiment of our invention at least one active of the extract is selected from this list.

Independent from these analytical results it was found, that extracts obtained by extracting parts of the Bauhinia species with a mixture of polar solvents and removing the solvent show excellent activities.

Therefore further embodiments of our invention are a method for producing a Bauhinia extract by a) extracting parts of the Bauhinia species with a mixture of polar solvents and b) removing the solvent and an extract obtainable by this method and extracts obtainable by this method.

The mixture of polar solvents preferably comprises water as one solvent and at least one other solvent selected from solvents less polar than water. A preferred list of such solvents includes methanol, ethanol, 1-propanol, 2-propanol, acetonitrile, aceton and ethyl acetate. A preferred solvent mixture contains 10-90% by volume water, especially preferred the mixture contains 30-70% by volume water.

The extraction process can for example be realized as follows: Dried leaves of Bauhinia are extracted with a mixture of ethanol and water with an ethanol content of 50% by volume. The extraction is done at a temperature between 20° C. and 90° C., preferably between 50° C. and 90° C. and especially preferred at about 70° C. After filtering off the solvent the process can be repeated to optimize the yield. The resulting extract is concentrated and the concentrate dried. The preferred method of drying is spray drying. Suitable conditions for the spry drying are given in example 1.

Advantages of the extracts according to the invention in hand in diabetes therapy are:

improvement of glucose tolerance

increase of the glucose clearance as seen by the slope of glucose disappearance rate

no effect on the insulin secretion in response to glucose.

The improvement of the glucose tolerance was shown to be very good during a therapy with administration of about 150 mg extract / kg body weight twice a day. These conditions seem to be an optimum for the therapy.

According to these advantages the use of the Bauhinia extract described herein as a medicament, especially for the production of a medicament with hypoglycemic activity and/or the production of a medicament suitable to influence the plasma glucose clearance and/or the production of a medicament suitable to influence the plasma glucose concentration, is another embodiment of the invention.

A further advantage is the antioxidant activity of the extracts, which is probably caused by the content of flavonoids in the extract. A further embodiment is therefore the use of a Bauhinia extract as an antioxidant.

Oxidative damage has been found to be associated with diabetes (Eds. L. Packer, P. Rösen, H. Tritscheler, G. King, A. Azzi; Antioxidants in diabetes management; New York—Basel; Marcel Dekker Inc., 2000). A role has been suggested for free radical damage and lipid peroxidation in the etiology of Type 2 diabetes mellitus (previously called NIDDM). There is evidence regarding the specific mechanisms by which free radicals could reduce insulin secretion (induce insulin deficiency) and impair insulin action (induce insulin resistance), leading to Type 2 diabetes.

There are some epidemiological data that individuals with higher levels of serum and tissues antioxidants (particularly high serum vitamin E) have lower risk of Type 2 diabetes. Oxidative stress is postulated to be increased in diabetic patients. Some of the causative agents of the increased stress in Type 2 diabetes are hyperglycemia, hypoinsulinemia, and an alteration of serum antioxidant activity.

Insufficiently controlled. diabetes is characterized by derangement of cellular defense mechanisms against oxygen radicals (J. Aaseth, O. W. Boe, “The biochemical basis of diabetic complications—a role of oxidative stress?” in: natural antioxidants and anticarcinogens in nutrition, health and disease, Cambridge, RSC, 1999, p.74-77). Particularly, in diabetic states glutathione level is greatly reduced in endothelial cells, retinal cells and other tissues. The levels of ascorbic acid are reduced extra- and intracellularly. The levels of serum vitamin E are reported to be low. Increased lipid peroxidation, increased generation of superoxide radicals, increased tissue levels of hydrogen peroxide, etc. are observed.

The impaired microcirculation, capillary hypoxia, and ischemic syndrome, present in most diabetic complications are associated with the production of reactive oxygen species. Among the possible mechanisms of the development of cellular consequences of oxidative stress is the activation of transcription factor NF-KB. The activation of this multiprotein complex, which resides as an inactive form in cytoplasm, is believed to play a pivotal role in the pathogenesis of diabetes.

The current strategy to combat diabetes focuses primarily on hyperglycemia control. However, only 30% of Type 2 diabetic patients can achieve the required glycemic control levels necessary to prevent further complications (Packer et al., 2000). Additional therapeutic approaches controlling oxidative stress can prevent micro- and neurovascular abnormalities, which are of relevance for major late diabetic complications, such as: nephropathy (renal disease, and eventually, kidney failure), retinopathy (retinal changes resulting in blindness),neuropathy (different types of nerve damage), and vasculopathy (atherosclerotic coronary and peripheral vascular disease).

Control of oxidative stress, along with blood sugar control, is a key approach to the treatment of diabetes. (Aaseth and Boe, 1999). Therefore diabetes therapy with the extract according to the invention in hand is especially advantagous for effects are achieved by using one composition.

Several antioxidants including vitamins C and E, α-lipoic acid, flavonoids, glutathione, carotenoids, coenzyme Q10, protein-bound zinc and selenium can be used in combination with the Bauhinia extracts to further improve the antioxidant activity of the drug.

The Bauhinia extracts described herein are useful as medicaments or as dietary supplements. Typical formulations contain 0.01 to 99% by weight of a Bauhinia extract as described herein.

The formulations may for example be in form of a powder, capsule, dragee or tablet and typically contain adjuvants necessary. to produce these application forms, which are known to the skilled man.

Preferred dietary supplements contain 0.01 to 99% by weight of additional supplements, such as vitamins, minerals, oligo elements, probiotics, prebiotics, fatty acids, flavonoids, polysaccarides, lipoic acid or plant extracts.

Preferred pharmaceutical formulations contain 0.01 to 99% by weight of additional antioxidants, preferably selected from the group containing vitamins C and E, α-lipoic acid, flavonoids, glutathione, carotenoids, coenzyme Q10, protein-bound zinc and selenium.

In one embodiment of the invention preferred formulations comprise further oral hypoglycemic agents (OHAs) such as sulfonylureas, mefformin and/or thiazolidinediones. Combination therapy with one or more of these agents and the Bauhinia extract of our invention is a preferred method of treatment.

The process to obtain the extract and the qualities of the extract are described in the following examples without limiting the invention in any manner.

### EXAMPLES

### Example 1

### Preparation of Bauhinia fortificata Dry Extract by Hydroalcoholic Extraction

1. Hydroalcoholic Extraction with Ethanol 50 (v/v)

1 kg of dry milled leaves of Bauhinia fortificata and 6 L of hydroalcoholic solution 50% (v/v) are put in a glass reaction flask and heated under reflux around 1 hour, with continuously stirring. Using a Büchner porcelain filter with a filter paper the extract is filtered.

In a second extraction step the same volume as the extract released of hydroalcoholic solution 50% is added to the residue in the glass reaction flask. The mixture is heated under reflux for 1 hour and filtered using a Büchner filter with a filter paper.

2. Pre-Concentration

The extract previously obtained is concentrated up to a total solids range between 5.0.% and 6.0% in a glass Evaporator/Concentrator under vacuum (500 mmHg).

3. Spray Drying

The concentrate is spray-dried in a Mini Spray Dryer B-191 (Büchi) under the following conditions:

Inlet temperature: 120° C.

Outlet temperature: 65-70° C.

Pump: 15%

Aspirator: 90%

Air flow: 400 ml/min

Pump flow: ˜220 ml/h

Cleaning temporizer: 2

Line pressure: 80 psi (5,5 bar)

4. Analytic

The extracts are characterized by High Pressure Liquid Chromatography (HPLC) and Thin Layer Chromatography (TLC).

### Example 2

### Oral Glucose Tolerance Test

Example 2a

The N0-STZ rat model is used to evaluate the efficacy of the extract of example 1. During 5 days an appropriate dose of the drug (table 1) is gavaged into 8 N0-STZ rats two times a day. Three hours after the last administration the Oral Glucose Tolerance Test (OGTr) is performed in awake rats by administering 2 g glucose/kg body weight. Blood samples are collected just before glucose administration (0 min) and after 30, 60 90 and 120 minutes.

The results (table 2 and FIG. 1) of the example according to our invention (example 22) are compared to results obtained with a Control (Example 23); results obtained for a group of rats administered with 100 mg/kg of mefformin 2 times a day during 5 days (example 24) and results obtained for a water-extract of young leaves of Bauhinia fortificata (example 25).

TABLE 1
drug and daily dose in the examples

| example | drug | daily dose |
|---|---|---|
| 22 | extract of example 1 | 2 × 150 mg/kg |
| 23 (control) | — | — |
| 24 | metformin | 2 × 100 mg/kg |
| 25 | water-extract of young leaves of Bauhinia | 2 × 500 mg/kg |
| | fortificata | |

TABLE 2
kinetics of glucose clearance: Plasma Glucose Level
[mg/dl] depending on time after administration
of 2 g glucose/kg body weight; −0 min: just before
administration

example

| | 22 | 23 (control) | 24 | 25 |
|---|---|---|---|---|
| time: −0 min | 122 | 143 | 143 | 145 |
| time: 30 min | 249 | 312 | 245 | 284 |
| time: 60 min | 252 | 281 | 242 | 269 |
| time: 90 min | 226 | 278 | 223 | 250 |
| time: 120 min | 202 | 253 | 207 | 227 |

TABLE 3
OGTT: Integration of the Plasma Glucose Level versus time (table 2)

| | example | | | |
|---|---|---|---|---|
| | 22 | 23 (control) | 24 | 25 |
| Area under | 27414 | 32057 | 26363 | 29688 |
| curve | | | | |
| error | 1253 | 1539 | 1167 | 1827 |
| deviation from | −14% | — | −18% | −7% |
| control (23) [%] | | | | |

The fasting plasma glucose concentration (table 4) is measured from blood samples obtained after a 2 hour fasting period on day 5 before the administration of glucose.

TABLE 4
Effect on fasting Plasma Glucose Concentration in % of
variation versus initial basal glycemia

example

| | 22 | 23 (control) | 24 | 25 |
|---|---|---|---|---|
| % | −28 | −14 | −31 | −20 |

The results obtained for example 22 are comparable to the results for example 24, while the results of example 25 show a weaker effect (table 3 and 4).

Example 2b

The N0-STZ rat model is used to evaluate the efficacy of the extract of example 1. During 5 days an appropriate dose of the drug (table 5) is gavaged into 8 N0-STZ rats. Three hours after the last administration the Oral Glucose Tolerance Test (OGTT) is performed in awake rats by administering 2 g glucose/kg body weight. Blood samples are collected just before glucose administration (0 min) and after 30, 60 90 and 120 minutes. The results are shown in table 6, 7 and FIG. 2.

TABLE 5
drug and daily dose in the examples

| example | drug | daily dose |
|---|---|---|
| 26 (control) | — | — |
| 27 | extract of example 1 | 2 × 150 mg/kg |
| 28 | extract of example 1 | 1 × 300 mg/kg |

TABLE 6
kinetics of glucose clearance: Plasma Glucose Level
[mg/dl] depending on time after administration
of 2 g glucose/kg body weight; −0 min: just before
administration

| | example | | |
|---|---|---|---|
| | 26 (control) | 27 | 28 |
| time: −0 min | 138 | 136 | 131 |
| time: 30 min | 284 | 247 | 282 |
| time: 60 min | 262 | 212 | 233 |
| time: 90 min | 221 | 186 | 191 |
| time: 120 min | 183 | 159 | 151 |

TABLE 7
OGTT: Integration of the Plasma Glucose Level versus time (table 6)

| | example | | |
|---|---|---|---|
| | 26 (control) | 27 | 28 |
| Area under | 11284 | 7382 | 9673 |
| curve | | | |
| error | 1217 | 999 | 1243 |
| deviation from | — | −35% | −14% |
| control (23) [%] | | | |

The area under the curve significantly decreases (−35% versus control; p<0.05; Dunett's test; table 7) when the extract is given twice daily at the dose of 150 mg/k (27). When the extract is given once a day (28) the area is slightly decreased but not significantly different from the control (26).

### Example 3

### Antioxidative Potential of the Eextract

The antioxidative potential of the extract of example 1 is measured as Trolox Equivalent Antioxidant Activity (TEAC-Assay), EC50 (DPPH-Assay) and relative antioxidant efficiency (RAE; Lipid-Assay) according to Halliwell,. B.; Eschbach, R.; Löhliger, J.; Aruoma, O.I.; Food. Chem. Toxicol. Vol 33, p. 601-67, 1995 (table 8).

TABLE 8
antioxidative potential of example 1

| method | result |
|---|---|
| TEAC-assay | TEAC = 0.12 (related to mg/l) |
| DPPH-assay | EC50 = 0.37 (related to mg/l to μmol/l) |
| Lipid-assay | RAE = 0.19 |

### FIGURES

FIG. 1: kinetics of glucose clearance: Plasma Glucose Level [mg/dl] depending on time after administration of 2 g glucose/kg body weight (0 min: just before administration) for examples 22-25

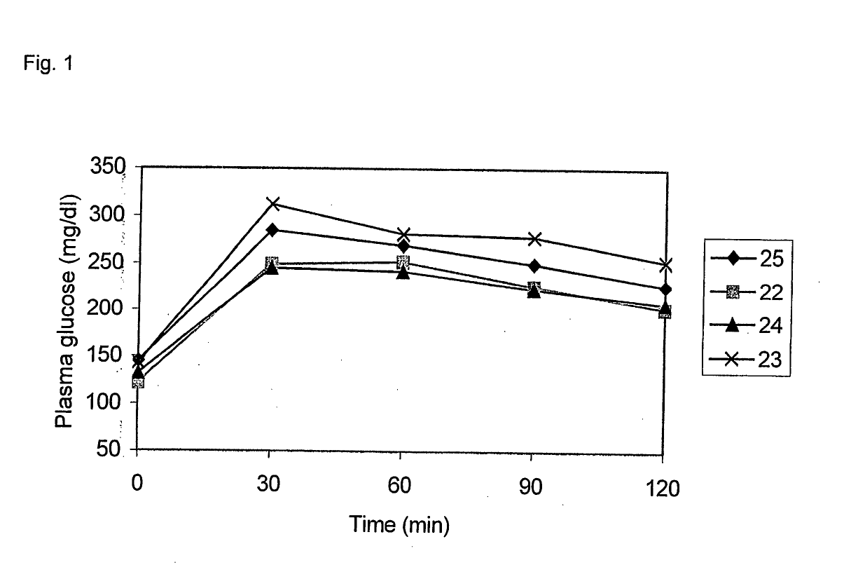

FIG. 2: kinetics of glucose clearance: Plasma Glucose Level [mg/dl] depending on time after administration of 2 g glucose/kg body weight (0 min: just before administration) for examples 26-28

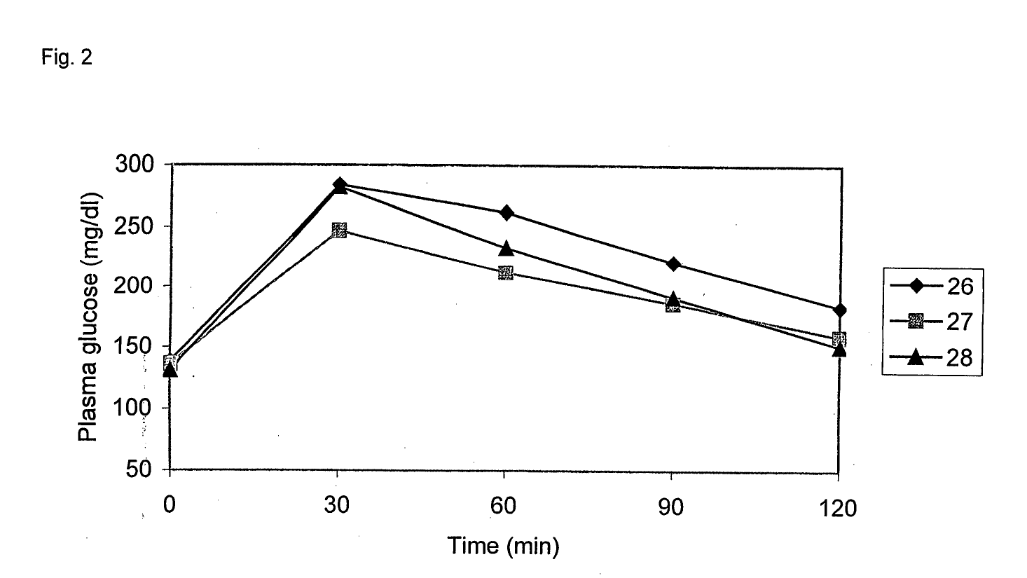

1. Plant extract with hypoglycaemic activity, characterized in that it is obtained from a Bauhinia species.

2. Extract according to claim 1, characterized in that the area under the curve—Plasma Glucose Concentration versus time- in the Oral Glucose Tolerance Test according to the N0-STZ Rat model decreases significantly.

3. Extract according to claim 2, characterized in that the area under the curve of Plasma Glucose Concentration versus time decreases by at least 10%, preferable at least 15%, even more preferred at least 20% and most preferred at least 35% versus control.

4. Extract according to one ore more of claims 1 to 3, characterized in that the extract, when applied to rats according to the N0-STZ Rat model, decreases the fasting Plasma Glucose Concentration versus initial Basal Glycemia significantly.

5. Extract according to at least one of the claims 1 to 4, wherein the Bauhinia species is selected from Bauhinia candicans, Bauhinia championii, Bauhinia fortificata (=Bauhinia forficata), Bauhinia manca, Bauhinia purpurea, Bauhinia racemosa, Bauhinia reticulata, Bauhinia tomentosa, Bauhinia variegate, Bauhinia cheilantha, Bauhinia guanensis, Bauhinia refusa, Bauhinia glauca, Bauhinia pauletia, Bauhinia ungulato, Bauhinia macrostachya and Bauhinia splendens, wherein the preferred species is Bauhinia fortificata (=Bauhinia forficata) and the leaves, preferably young leaves are the part of the plant used in extraction.

6. Extract according to at least one of the claims 1 to 5 with hypoglycaemic activity comprising at least two actives, where at least one of the actives is supposed to be a flavone or flavonoid, such as apigenin, apigenin-7-O-glucoside, isoquercetin (isoquercitrin), kaempferol-3-rutinoside, kaempferol-3-galactoside, kaempferol-3-rhamno-glycoside, kaempferol-3-glucoside (astragalin), naringenin-4′-rhamnoglucoside, naringin, quercetin, quercetin-3,7-O-α-dirhamnoside, quercitrin, 5,7-dimethyl-ether-4′-rhamnoglycoside, rutin, 5,7-dihydroxyflavanone-4′-O-L-rhamnopyranosyl-β-D-glucopyranoside (naringenin aglycone), preferably at least one active is selected from quercetin, isoquercetin or rutin.

7. Extract according to at least one of the claims 1 to 6, obtainable by a) extracting parts of the Bauhinia species with a mixture of polar solvents and b) removing the solvent.

8. Extract according to claim 7, wherein the mixture of polar solvents comprises water as one solvent and at least one other solvent selected from methanol, ethanol, 1-propanol, 2-propanol, acetonitrile, aceton and ethyl acetate and a preferred solvent mixture contains 10-90% by volume water, especially preferred the mixture contains 30-70% by volume water.

9. Method for producing a Bauhinia extract by a) extraction parts of the Bauhinia species with a mixture of polar solvents and b) removing the solvent.

10. Use of a Bauhinia extract according to at least one of the claims 1 to 8 as a medicament.

11. Use of a Bauhinia extract according to at least one of the claims 1 to 8 as an antioxidant.

12. Use of a Bauhinia extract according to at least one of the claims 1 to 8 for the production of a medicament with hypoglycaemic activity.

13. Use of a Bauhinia extract according to at least one of the claims 1 to 8 for the production of a medicament suitable to influence the plasma glucose clearance.

14. Use of Isoquercetin for the production of a medicament with hypoglycaemic activity.

15. Formulation containing 1 to 99% by weight of a Bauhinia extract according to at least one of the claims 1 to 8 or an extract produced by a method according to claim 9.

16. Dietary supplement according to claim 15, containing 0.01 to 99% by weight of additional supplements, such as vitamins, minerals, oligo elements, probiotics, prebiotics, flavonoids, fatty acids, polysaccarides, lipoic acid or plant extracts.

17. Pharmaceutical formulation according to claim 15, containing 0.01 to 99% by weight of additional antioxidants, preferrably selected from the group containing vitamins C and E, α-lipoic acid, flavonoids, glutathione, carotenoids, coenzyme Q10, protein-bound zinc and selenium.

18. Pharmaceutical formulation according to one or more of the claims 15 and 17, characterized in, that it comprises further oral hypoglycemic agents (OHAs) such as sulfonylureas, mefformin and/or thiazolidinediones.
